(19) United States
(12) Patent Application Publication
(10) Pub. No.: US 2008/0171057 A1
(43) Pub. Date: Jul. 17, 2008

(54) Reagents, Devices, and Methods For Proteomic Analysis With Applications Including Diagnostics, Vaccines, Quality Control and Research

(75) Inventor: Geoffrey William Hoffmann, Vancouver (CA)

Correspondence Address: VERMETTE & CO., SUITE 320 - 1177 WEST HASTINGS STREET, VANCOUVER, BC, V6E2K3, omitted

(73) Assignee: NETWORK IMMUNOLOGY INC., Vancouver, BC (CA)

(21) Appl. No.: 11/568,183

(22) PCT Filed: Apr. 19, 2005
(86) PCT No.: PCT/CA2005/000606

§ 371 Date: Mar. 11, 2008

Related U.S. Application Data
(63) Continuation-in-part of application No. 11/049,964, filed on Feb. 4, 2005.
(60) Provisional application No. 60/563,819, filed on Apr. 21, 2004.

(51) Int. Cl. A61K 39/00; A61P 35/00; A61P 37/00; G06F 17/00

(52) U.S. Cl. 424/184.1; 708/132

(57) ABSTRACT

The invention describes methods for proteomic analysis involving the mapping of samples in N-dimensional shape space. The applications include the classification of samples on the basis of the three-dimensional shapes of substances they contain. A panel of P (>>1) reagents, with P≧N, called X(j), with j=1 to P, is used, The binding strength of each of the X(j) reagents to each other is a P×P matrix. This matrix is used to define another set of P reagents called Y(j), with j=1 to P, each of which is a linear combination of the X(j) reagents and each of which is complementary to one of the X(j) reagents. N of the X(j) reagents together with the corresponding Y(j) reagents are used to define a shape space that has N approximately orthogonal axes. The definition of these axes facilitates classification of samples. Methods for measuring similarity between pairs of samples and between sets of samples in the context of the set of N reagent pairs X(j) and Y(j) with j=1 to N are described. Applications include classification of samples, quality control, methods of diagnosis, and formulation of vaccines.

This application claims the benefit of previously filed Provisional Patent Application No. 60/563,819, filed on Apr. 21, 2004.

### I. FIELD OF THE INVENTION

This patent application describes methods of proteomic analysis and synthesis of samples that can be simple or complex mixtures of substances. One of the methods is a method of classification of samples, that can be used for example in the quality control of manufactured or biological goods. The methods include methods for the analysis of immune system V (variable) regions, with the classification of individuals with respect to various diseases (diagnosis). The diagnostic methods include measurements of binding of reference sets of reagents to immune system V regions. Immune system V region proteomics is important because the immune system V region repertoire is changed or “skewed” in many diseases, including cancer, autoimmune diseases and graft versus host disease. O'Neill, 1991, Cell. Immunol., 136, 54-61; Wucherpfennig et al. 1992, J Exp Med., 175, 993-1002; Imberti et al. 1991, Science 254, 860-862; Rebai et al. 1994, PNAS 91, 1529-33. This skewing opens possibilities for innovations in diagnostic testing. The methods include methods for preventing and/or treating diseases for which the skewing of the repertoire of immune system V regions is well characterized. These methods involve an immunization or immunizations that are tailored to reverse the particular skewing.

### II. BACKGROUND TO THE INVENTION

This invention includes the ability to classify a wide range of samples that may be simple or complex. It emerged in the context of classifying vertebrates with respect to various diseases on the basis of immune system V regions in biological samples.

A full proteomic description of the specific (V region) components of a particular immune system would constitute a list of the concentrations of each of millions of lymphocytes, antibodies and specific T cell factors, together with the isotypes, amino acid sequences and three-dimensional structures of the corresponding V regions. Even with the spectacular advances that are currently being made in proteomics, such a description is not a realistic goal, and even if it were, achieving it may not be particularly useful. Each individual has his or her own set of V regions, due to different V region genes, different MHC (major histocompatability complex) genes that affect the expressed repertoire of T cells, and different histories of exposure to a wide range of antigens. Furthermore, different somatic mutations in each individual contribute significantly to the generation of the V region repertoire.

One recent approach to diagnostic proteomics is the SELDI-MS technology coupled to pattern recognition software. Hitt et al. United States Patent Application Publication, Pub. No. US 2003/0004402 A1. This is not suited for V region proteomics because it is based on mass differences between molecules, and while (for example) IgG antibodies with different V regions can have slightly different masses, each person has a unique spectrum of antibodies.

On the other hand, ELISA (enzyme-linked immunosorbent assay) technology and Radio Immune Assay (RIA) technology are available that are suitable for V region proteomics.

This patent application describes a method for proteomic analysis that builds on the previously defined concept of serological distance coefficients. Hoffmann et al. 1989 Immunology Letters, 22, 83-90. Experimentally measurable similarity coefficients S[A,B|C] specify the extent to which a pair of substances, A and B. are similar in the context of a diverse reagent, C. The definition of S[AB|C] is the fraction of C that binds both A and B divided by the sum of (i) the fraction that binds A but not B, (ii) the fraction that binds B but not A and (iii) the fraction that binds both A and B. The value of S[A,B|C] is then necessarily a number between zero and one. This definition was applied also to similarities between complex mixtures of substances, such as the antibodies of two serum samples, A and B. A “distance coefficient” D[A,B|C] between two sera, A and B, in the context of C, was defined as one minus the similarity coefficient in the same context. The experimental measurement of these coefficients, and their possible use in the diagnosis and prognosis of disease conditions was described.

This invention invokes the concept of shape space. An N-dimensional shape space has been discussed by Perelson et al. 1979, J. theor. Biol. 81, 645-667, and a formulation that permits an experimental determination of the dimensionality of a shape space has been described by Lapedes et al. J. theor. Biol. 2001, 212, 57-69. The N-dimensional shape space of this invention is different from both of these; the different shape spaces are contrasted near the end of the detailed description of the invention.

The antibody repertoire of the immune system is regulated by the T cell repertoire. The T cell repertoire in turn is selected by self antigens, including most notably MHC (Major Histocompatability Complex) antigens, but possibly also the many self antigens that are much less polymorphic than MHC antigens. The impact of non-polymorphic self antigens on the T cell repertoire would not be seen in the kinds of experiments that demonstrate the high level of polymorphism in MHC antigens. A plausible evolutionary constraint on self antigens is that they should consist of a “balanced” set, such that for any self antigen impinging on the immune system and stimulating one set of clones, there are other self antigens that stimulate complementary clones. The immune system may itself (in addition) dynamically establish symmetry between each shape and complementary shapes in V region repertoires. This concept leads to the idea of a high level of similarity in the expressed antibodies repertoires of young, healthy individuals of different species, in the sense of them all being “balanced” repertoires in this respect. Among other applications, this invention will enable the concept of balanced repertoires, and hence similar repertoires even in healthy individuals of different species, to be experimentally tested.

The immune system is a highly sensitive system that can be modulated by very small amounts of antigens and antibodies. Experiments in mice and rats show that the specific response of the system to a particular antigen can be significantly decreased by injections of antigen as low as picograms or even less. Shellam 1969 Immunol. 16, 45-56; Ada et al. 1968 Proc. Nat. Acad. Sci. (USA), 61, 566-561. A response consisting of antibodies with a particular idiotype can be suppressed by an injection of 10 to 100 ng of antiidiotypic antibody. Eichmann 1974 Eur. J. Immunol., 4, 296-302. The injection of nanogram amounts of monoclonal IgM antibody can induce the production of antibodies of the same specificity. Forni et al. 1980. Proc. Nat. Acad. Sci. (USA) 77, 1125-1128. The genetic manipulation of adding a single heavy chain gene, that is a marker of a particular idiotype, to the genome of a mouse results in the-production of antibodies with the same idiotype, but using other genes. Weaver et al., 1985. Cell, 45, 247-259. It would seem that such manipulations of the immune system would make a marked difference to the state of the system only if it is normally precisely balanced. Only then might one expect that such very small perturbations can shift the state of the system significantly. Hence such findings suggest that a dynamically maintained balance between shapes and complementary shapes is a basic feature of the V regions of the immune system. Various diseases then correspond to various forms of a loss of balance in the system.

### BRIEF DESCRIPTION OF THE DRAWINGS

Further features and advantages will be apparent from the following Detailed Description of the Invention, given by way of example, of a preferred embodiment taken in conjunction with the accompanying drawings, wherein:

FIG. 1: The reagents X(1) and Y(1) are complementary to each other and define an axis in shape space, and the reagents X(2) and Y(2) define a second axis. The coordinates of sample i are determined by measuring the amount of binding of the reagents X(1), Y(1), X(2) and Y(2) to the sample. Here sample i binds more to X(1) than to Y(1) and more to X(2) than to Y(2). Hence it is more similar to Y(1) than to X(1) and more similar to Y(2) than to X(2).

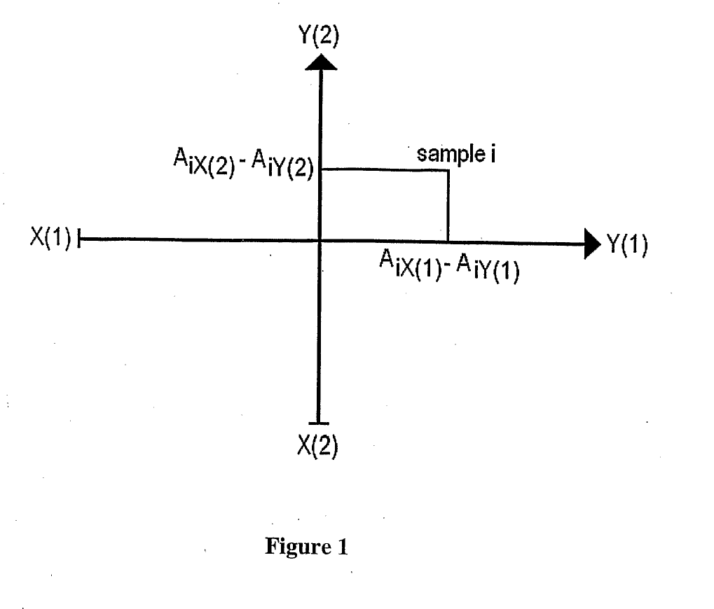

FIG. 2: The coordinate of a sample with Proteomic Analyser point Aᵢ on the axis defined by A₁ₐᵥ and A₂ₐᵥ is xᵢ, where

$${x}_{i} = \frac{1}{2 c} ( {a}_{i}^{2} - {b}_{i}^{2} + {c}^{2} ) ,$$

c is the Euclidean distance from A₁ₐᵥ to A₂ₐᵥ, aᵢ is the Euclidean distance from A₁ₐᵥ to Aᵢ, and bᵢ is the Euclidean distance from A₂ₐᵥ to Aᵢ.

FIG. 3: Average absorbances A_{HavX(j)}, A_{DavX(j)}, A_{HavY(j)} and A_{DavY(j)} plotted on the A_{X(j)} and A_{Y(j)} axes. The average disease state, A_{DavX(j)Y(j)}, and the average healthy state, A_{HavX(j)Y(j)}, from the perspective of the X(j) and Y(j) pair of reagents is shown. (Note that this is a different perspective on the N-dimensional shape space from that of FIG. 1.)

### III. DETAILED DESCRIPTION OF THE INVENTION

This invention includes methods of classification of samples, and these methods lead to applications including quality control and methods for diagnostics and vaccine formulation. The invention utilises a number P (>>1) of reagents, rather than a single diverse reagent, where P≧N and N is the number of dimensions of a shape space with approximately orthogonal axes. Each of the reagents can be an individual substance or a mixture of substances. This produces a much larger data set than using a single diverse reagent, but it is still a very small set compared with, for example, the complete listing of V regions and their concentrations mentioned above. The result is a measure of similarity between substances or mixtures of substances (“samples”) based on the N-dimensional shape space, and is a more powerful tool for multiple applications, including applications to diagnostics and vaccines. diagnostics and vaccines. The new approach also has the advantage that it eliminates the need to do absorptions of the diverse reagent C, which was the most labour-intensive part of the determination of serological distance coefficients as previously described.

The members of the panel of P reagents are selected on the basis of being diverse and having well-defined, reproducible three-dimensional shapes and the constraint that the N shape space axes are optimally orthogonal. They may, for example, include, but are not restricted to being, normal human proteins and proteins of one or more other species.

We consider first the case that P=N. We denote the reagents of this panel by X(j) (with j=1 to N), and use them all most simply (but not necessarily) at a standard concentration C₀. We measure the binding (relative affinity) of each of these reagents to each other using, for example, an ELISA or an RIA. This produces a matrix K with elements Kⱼₖ (j=1, N, k=1, N).

We next define N new reagents, that we denote as Y(j), (j=1, N). Each of the Y(j) reagents is made up of a linear combination of the X(j) reagents, with the amount of the k^{th} component being proportional to Kⱼₖ. Those components that have strong binding to X(j) are present at a high concentration in Y(j), while those with little or no binding are included at a low or zero concentration, For each X(j) there is a corresponding Y(j), with j=1 to N. There are two possible ways of normalizing the concentrations of the Y(j) reagents to establish a symmetry between the X(j) reagents and the Y(j) reagents. One is to make the total concentration of the components of Y(j) such that the binding signal obtained for Y(j) binding to X(j) (in the case of an ELISA assay, with Y(j) binding to X(j) on the plate), in the linear range of the assay, is equal to the converse binding signal (binding of X(j) to Y(j), also in the linear range of the assay). The other method is to simply set the total concentration of each Y(j) equal to C₀. The former method leads to the definition of a convenient virtual N-dimensional origin for the shape space, namely a hypothetical sample to which X(j) and Y(j) bind equally in the assay, for all values of j.

Each pair of reagents X(j) and Y(j) are complementary to each other and are thus opposite poles of an axis in the N-dimensional shape space. Together they define an axis in that space called the X(j)/Y(j) axis. We measure the binding of each X(j) reagent (j=1, N) to each Y(k) (k=1, N) reagent. This produces the N×N matrix J with elements Jⱼₖ. On the basis of mass-action, and subject to linearity of the assay, the expected relative values of the elements of J are

$${J}_{jk} = {\sum }_{l = 1}^{N} {K}_{jl} {K}_{lk} \qquad ( 1 )$$

The diagonal elements of this matrix specify the level of binding between the reagents X(j) and Y(j), that have been specifically tailored to be complementary to each other. Hence their mutual binding will produce a strong binding signal, while there will be a relatively weak signal for off-diagonal terms. Thus J is an approximately diagonal matrix. The interpretation of this feature is that the N X(j)/Y(j) shape space axes are approximately mutually orthogonal.

We now consider samples, for example biological samples containing immune system V regions obtained from an individual i. These samples may be, for example but not exclusively, serum, T-lymphocyte extracts, B-lymphocyte extracts, saliva or urine. We measure the binding of each of the reagents X(j) (j=1 to N) to each of the samples, again using for example an ELISA or an RIA. For each sample we thus obtain N binding signals A_{iX(j)}.

We repeat this process using the set of N complementary reagents, Y(j). We measure the binding of each Y(j) reagent to components in the sample i, to obtain the values A_{iY(j)} (measured) for j=1 to N. Subject to an assumption concerning linearity of the assay, we can however also compute expected relative values of A_{iY(j)} according to:

$${A}_{iY ( j )} ( expected ) ∝ {\sum }_{k = 1}^{N} {A}_{iX ( k )} {K}_{kj} \qquad ( 2 )$$

The results of these summations are then normalized such that the average of the computed values of A_{iY(j)} is the same as the average of the measured A_{iX(j)} over j=1 to N. Hence, remarkably, we can have the benefit of an analysis in terms of the N X(j)/Y(j) axes in shape space without needing to prepare the Y(j) reagents, and without making measurements on all our samples using them! This is because the values of the Aᵢⱼ together with the K matrix values already contain all the physical information. On the other hand, by including the actual measurement of A_{iY}(j) using Y(j) reagents we have a technology that is more robust, because the individual measurements are then automatically screened for self-consistency. This is analogous to sequencing both strands of DNA, in which case any sequencing errors are immediately revealed, since one sequence predicts the other. The inclusion in the technology of the measurements using Y(j) reagents is expected to be done at only a low additional cost. To the extent that the results differ, the best estimate of each A_{iY(j)} may be obtained by taking the mean of the measured and computed values.

The difference A_{iX(j)}−Ar_{iY(j)} is a coordinate for the sample i on the X(j)/Y(j) axis, that can be either positive or negative, and will be denoted as Aᵢⱼ. It specifies whether the sample i is more X(j)-like (Aᵢⱼ<0) or more Y(j)-like (Aᵢⱼ>0). There are N such coordinates (j=1 to N) for each sample. The set of N coordinates Aᵢⱼ with j=1 to N is called the Proteomic Analyser point (“PA point”) for the sample i and in the case of a biological sample is a PA point for the individual or organism from whom or from which the sample was derived. This set of N coordinates for the sample i will be denoted by “Aᵢ”.

The orthogonality of the shape space can be increased by using more reagents (“P reagents”) than the number of dimensions of the shape space (N) as follows. We use the set of P reagents X(j), j=1 to P, where P>N, and measure the P×P matrix “K^{P}” with elements “K^{P}ᵢⱼ” (i=1 to P, j=1 to P) being the binding signals of each of the reagents to each other as before for the matrix K. We formulate a full set of reagents Y(j) (j=1 to P), using the full set of P X(j) reagents and the matrix K^{P} to determine the relative concentration of each X(j) reagent in each Y(j) reagent. That is, each Y(j) reagent, for j=1 to P, consists of a weighted mixture of the P reagents, with the relative amount of the k^{th} component being proportional to K^{P}ⱼₖ, for k=1 to P. We measure the binding of each of the X(j) reagents to each of the Y(j) reagents to obtain the P×P matrix J^{P}. We then select the N X(j) and Y(j) reagent pairs that have the largest ratio of the diagonal elements of J^{P} to the mean of the corresponding off-diagonal elements (terms in the same row and the same column). These N X(j)s and Y(j)s are then used in the experimental measurement of PA points for an N-dimensional shape space as already described. For these N X(j) and Y(j) reagents we have a K matrix and a J matrix as before. Then we obtain a set of N coordinates for the sample i denoted by “Aᵢ” using this set of X(j) and Y(j) reagents as before. For a single shape space axis at least two reagents are needed, and making P=2N provides an additional degree of freedom for each shape space axis.

The above methods are designed to have no a priori bias or preference for any shape space axis over any other. This is desirable, since the goal is to map samples in a shape space that is as symmetrical as possible with respect to the universe of shapes. The result is that the magnitudes of the diagonal elements of J do not differ greatly from each other. These methods are therefore preferred to strategies that may achieve orthogonality of the N axes in a more managed way, and in the process result in some of the diagonal elements of J being much larger than others. Criteria for judging which methods of selection of the X(j) and Y(j) reagents are most successful include the resulting degree of diagonal dominance of J and the amount of uniformity in the magnitudes of the diagonal elements of J.

The first aspect of the invention is thus providing the ability to experimentally map samples, that can be either simple (few component substances) or complex (many component substances) in an N-dimensional shape space. This mapping is useful because it permits one to measure the distance in the N dimensional shape space between different samples, and permits the classification of samples based on where they map relative to each other in the space. If a category of samples maps clearly to within a defined region of the N dimensional space, and a sample maps clearly outside of that region, the mapping can be used to exclude that the sample belongs to that category. More generally, the mapping of groups of samples in different categories in the N dimensional shape space (for example giving mean, and standard deviation for the distribution in each of the N dimensions for each category) permits straightforward statistical methods to be used to compute relative probabilities of unclassified samples belonging to the various categories to be estimated, based on where they map in the N dimensional shape space. This means that a central aspect of the invention is that it provides the basis for an ability to classify samples with respect to categories.

An important application of this ability to classify samples with respect to categories is the diagnostic aspect of the invention, in which the different categories include sets of samples from individuals that are healthy and sets of samples from individuals with any of a variety of diseases. Each disease is expected to be characterized by Proteomic Analyser points within disease-specific regions, while healthy individuals are expected to be characterized by different fingerprints. For this application the samples contain immune system variable regions (“V regions”), and the binding of the reference set of reagents to immune system V regions is measured.

The diagnostic aspect leads to a vaccine aspect of the invention, in which the adaptive property of the immune system makes it possible to modify the immune system, and move the Proteomic Analyser point for the V regions of a person with a given disease (or whose Proteomic Analyser point is on a trajectory towards a given disease) back towards the Proteomic Analyser point that is characteristic of a healthy person, or (in a personally customized aspect of the invention) toward the Proteomic Analyser point of that person when he or she was healthy. The same set of reagents that are used to measure the Proteomic Analyser point are used to stimulate the immune system, such that it moves in the direction back towards a Proteomic Analyser point characteristic of the healthy state. For different diseases, different (calculable) recipes (lists) of the same set of reagents are used.

The ability to classify samples with respect to categories leads to the possibility of quality control for many goods, including for example agricultural goods. Extracts of samples of meat can have their Proteomic Analyser points measured and checked for consistency. Suppliers and purchasers of such items as grains and yeast (for making bread) may similarly find it advantageous to have the items certified to have Proteomic Analyser points within a specified range of what they know, from experience, to be satisfactory values. The manufacturers of breakfast cereals may find it useful to monitor the Proteomic Analyser points of batches of their products. A farmer may find it advantageous to measure Proteomic Analyser points of soil samples, and determine which Proteomic Analyser points for the soil samples correlate with good yields for various crops.

In light of these examples of potential applications, the potential utility of being able to measure Proteomic Analyser points is evident.

Mapping samples in an approximately orthogonal N-dimensional shape space leads to a method for classifying a wide range of samples with respect to a wide range of categories. We consider an unclassified sample U that we want to classify with respect to Q categories, where Q is an integer equal to or greater than 2, and with each of the categories labelled by a value of q, where q=1 to Q. We select M₁ samples that are known by conventional criteria to belong to the category 1, select M₂ samples that are known by conventional criteria to belong to the category 2, and in general select M_{q} samples that are known by conventional criteria to belong to the category q, thus using a total of Q sets of samples that have been classified using conventional criteria. We map the samples in each category in an N-dimensional, approximately orthogonal shape space, giving coordinates A_{qij} with q−1 to Q, i=1 to M, and j=1 to N and let these PA points be denoted by A_{qi}. We map the unclassified sample U in the same N-dimensional shape space, giving coordinates A_{Uj}, with j=1 to N and we let this PA point be denoted by A_{U}.

We compute the N average Proteomic Analyser coordinates A_{qav(j)} for j=1 to N and q=1 to Q, of the M_{q} samples in each of the Q categories (their average PA point) as

$${A}_{qav ( j )} = \frac{1}{{M}_{q}} {\sum }_{i = 1}^{{M}_{q}} {A}_{qij} \qquad ( 3 )$$

and designate these average PA points “A_{qav}”, with q=1 to Q.

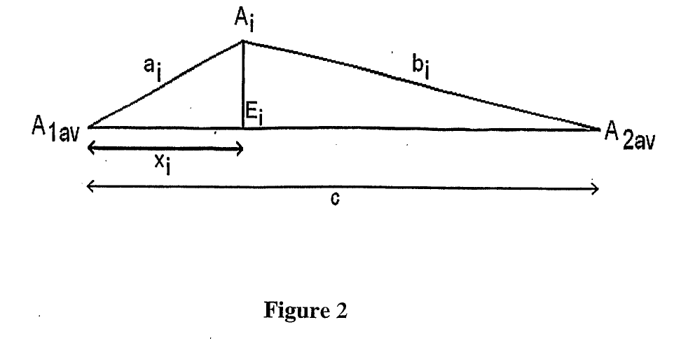

We select two of the sample set averages Aq to define a new axis in shape space (FIG. 2). The first of these is typically a reference prototype category, and we make this category 1. For example, in the case of the application of this method to diagnostics, this category is a set of samples from young, healthy individuals. Let the second category, be category 2. Our first computation is to determine whether the PA point of the sample U and the PA points of the set of samples in categories 1 and 2 are such that we are able to exclude that the sample U belongs to either or both of the categories 1 and 2. The two categories have sample set averages A₁ₐᵥ and A₂ₐᵥ, where each of these points have N coordinates A₁ₐᵥ₍ⱼ₎ and A₂ₐᵥ₍ⱼ₎, with j=1 to N. We calculate the Euclidean distances between the sample averages A₁ₐᵥ and A₂ₐᵥ according to

$$c = \sqrt{{\sum }_{j = 1}^{N} {( {A}_{1 av ( j )} - {A}_{2 av ( j )} )}^{2}} \qquad ( 4 )$$

and let this be designated “c” as shown in FIG. 2. We let the data points A_{qi} (for q=1 and q=2, i=1 to M₁ and i=1 to M₂ respectively) and Au be collectively referred to as Aᵢ. We let the Euclidean distance from each Aᵢ to A₁ₐᵥ be designated “aᵢ” and let the Euclidean distance from Aᵢ to A₂ₐᵥ be designated “bᵢ” as shown in FIG. 2. We draw a line from Aᵢ to the A₁ₐᵥ/A₂ₐᵥ axis at right angles to the A₁ₐᵥ/A₂ₐᵥ axis; this intersects the axis at a point designated Eᵢ, as shown in FIG. 2. The distance from A₁ₐᵥ to Eᵢ is designated xᵢ. We compute the xᵢ for all of the data points Aᵢ as

$${x}_{i} = \frac{1}{2 c} ( {a}_{i}^{2} - {b}_{i}^{2} + {c}^{2} ) \qquad ( 5 )$$

We compute the mean and standard deviation of the xᵢ for samples in the category 1 and category 2 and let them be denoted by μ₁(xᵢ), μ₂(xᵢ), σ₁(xᵢ) and σ₂(xᵢ) respectively. We denote the value of xᵢ for the unclassified samples by xᵢ(U).

In the context of the model that the distributions of values of xᵢ for samples within each of the two categories is approximately normal, we calculate the z statistic, z_{U(q)} (q=1 and q=2), for the xᵢ of the unclassified sample U relative to the distribution of xᵢ values for samples in each of the categories 1 and 2,

$${z}_{U ( q )} = \frac{{x}_{i} ( U ) - {μ}_{q} ( {x}_{i} )}{{σ}_{q} ( {x}_{i} )} \qquad ( 6 )$$

From these computed statistics for xᵢ with q=1 and 2, we determine whether the unclassified sample U can be excluded from the categories 1 or 2, and if so, from which categories and with what level of confidence. We repeat this process with q=1 and 3, then 1 and 4, and so on to 1 and Q, to determine whether the samples can be excluded from each of the other categories, and if so, with what level of confidence, with category 1 in each case as the reference category. This process can also be implemented with a different category (q not equal to 1) as the reference category.

We can use a second approach to compute relative probabilities for the sample belonging to each of the various categories. The distributions of the coordinates of the samples in the database in each of the N dimensions defined by the N reagent pairs X(j) and Y(j) is used. We begin with using the N mean coordinates of each group, A_{qav(j)}, to compute the standard deviations σ_{qj} (j=1 to N, q=1 to Q) for each of the N coordinates of the M_{q} samples in each group as

$${σ}_{qj} = \sqrt{\frac{{\sum }_{i = 1}^{{M}_{q}} {( {A}_{qij} - {A}_{qav ( j )} )}^{2}}{{M}_{q}}} \qquad ( 7 )$$

We use the values of the coordinates A_{Uj} (j=1 to N) (the components of A_{U}), the computed values of the standard deviations σ_{qj}, and the model that the values of A_{qij} for a given category (fixed value of q), a given value of j, and i=1 to M_{q} are normally distributed about the mean A_{qav(j)}. The normal distribution probability for the jth coordinate of the unclassified sample having the value A_{Uj} is given by

$$F ( {A}_{Uj} , {A}_{qav ( j )} , {σ}_{qj} ) = \frac{1}{{σ}_{qj} \sqrt{2 π}} exp ( - \frac{{{A}_{Uj} - {A}_{qav ( j )} )}^{2}}{{M}_{q}} ) \qquad ( 8 )$$

We compute the ratio [P_{U1}/P_{U2}]ⱼ of the probability that the unclassified sample U belongs to the category 1, to the probability that it belongs to the category 2, based on the data for these two categories for the jth shape space axis according to

$${[ {P}_{U 1} / {P}_{U 2} ]}_{j} = \frac{F ( {A}_{Uj} , {A}_{1 av ( j )} , {σ}_{1 j} )}{F ( {A}_{Uj} , {A}_{2 av ( j )} , {σ}_{2 j} )} \qquad ( 9 )$$

We then compute the joint probability ratio using the data for all N (approximately orthogonal, hence approximately independent) axes in shape space, [P_{U1}/P_{U2}]_{all N axes}, as the product from j=1 to j=N of the probabilities for each of the axes [P_{U1}/P_{U2}]ⱼ according to

[P_{U1}/P_{U2}]_{all N axes}=[P_{U1}/P_{U2}]₁[P_{U1}/P_{U2}]₂[P_{U1}/P_{U2}]₃ . . . [P_{U1}/P_{U2}]_{N} (10)

We can use this same procedure for computing the probability ratio for the sample i belonging to each of the other Q-2 categories relative to category 1. We can also compute in the same way other relative probabilities for the sample belonging to various categories, for example the probability that a sample belongs to category 5 relative to the probability of it belonging to category 6. The more samples we have in each category, the more accurately we can determine the means and standard deviations for each category with respect to each of the N axes, and the more accurate the classification results will be.

### A Proteomic Diagnostic Method

The above method of classification can be used as a diagnostic method. A premise of the diagnostic aspect of the invention is that immune system V regions in healthy individuals map to a limited, characteristic region in the N-dimensional shape space. This aspect is demonstrated using the Proteomic Analyser itself. Some diseases, such as autoimmune diseases, correspond to particular modes of aberration or collapse of the immune system network of V regions, and immune system V regions in samples from people with each of these diseases map to different, disease-specific regions of the N dimensional shape space. Some diseases are characterized by a disease-specific set of aberrant self antigens (as in the case of cancers) and are also associated with characteristic, disease-specific perturbations of the PA point relative to the healthy, young PA point for the individual. For this application category 1 refers to a set of samples from healthy, preferably young individuals. The other categories are sets of samples from people that have been classified to have various diseases.

The combination of the two classification processes as described above provides a diagnosis comprising both a list of diseases that are excluded and a list of relative probabilities for diseases. For example, a diagnosis may be that each of ten forms of cancer, Alzheimer's disease and Creutzfeldt-Jakob disease are excluded with confidence levels of 95% or higher, while lupus, diabetes and osteoarthritis are not excluded, and with the individual being one hundred times more likely to have lupus than being healthy, fifteen times as likely to have lupus as diabetes and five times as likely to have lupus as osteoarthritis.

So far we have included all of the N reagents in the analysis. We do not need to do this. For the diagnosis of a particular disease or condition we can instead include only those reagents that optimise specificity, sensitivity and simplicity, either individually or jointly.

An advantage of this diagnostic method over the precursor serological distance coefficient method is the fact that it eliminates the need to do absorptions, which was the most labour-intensive part of that earlier method.

Another advantage is that this diagnostic method is based on N-dimensional vectors, with N>>1 as opposed to the 2-dimensional map of the previously published serological distance coefficient diagnostic method, that utilised a single diverse regent. This means that the method provides more specific diagnoses. N-dimensional vectors with N>>1 contain much more precise information than 2-dimensional vectors.

In addition to the actual position in N-dimensional shape space, the direction of movement of the coordinates in shape space for an individual from a healthy state towards coordinates characteristic of having a particular disease is indicative of progression towards having that disease.

An example of a disease that has historically been difficult to diagnose is systemic lupus erythematosus (SLE). The definition of SLE of 1982 (Tan et al., Arthritis Rheum. 25, 1271-1277, 1982) includes eleven classes of criteria, with multiple alternative sub-criteria for five of these, such that there is a total of twenty criteria. An individual is defined as having lupus if he or she has four or more of the eleven classes of criteria. The Proteomic Analyser method can be used to identify people who have lupus or whose immune systems are on a trajectory towards having lupus.

### Application To Vaccine Formulation

In addition to its diagnostic role, the formalism and method developed here is useful for the formulation of highly specific multi-component proteomic perturbations to the immune system that function as preventive and/or therapeutic vaccines. This is the case when the diagnosis involves measurements of the binding of the set of reagents to immune system V regions. The diagnosis then measures skewing of the immune system repertoire of V regions relative to the repertoire of healthy individuals, and a stimulus consisting of a combination of the X(j) and Y(j) reagents can be tailored to correct the skewing.

The V region repertoire of an individual can be changed by stimulation with the X(j) and Y(j) reagents. This involves the process of clonal selection, in which cells with specific (V region) receptors that are complementary to a substance are stimulated by that substance to proliferate. Since each X(j) is complementary to the corresponding Y(j), cells with V region receptors that are complementary to the X(j) reagents will be called “Y(j) cells” and cells with V region receptors that are complementary to the Y(j) reagents will be called “X(j) cells”. The process of correcting skewing in the system involves a computed recipe for the stimulation of X(j) cells by the Y(j) reagents and stimulation of X(j) cells by the Y(j) reagents.

We use a set of M_{D} samples containing immune system V regions from individuals who have been classified to have a given disease (the “D set”), and another set of M_{H} samples containing immune system V regions from healthy individuals (the “H set”). We obtain M_{H}N binding signals A_{H(i)X(j)} of the X(j) reagents to immune system V regions for the healthy group, where i is an index for the sample that goes from 1 to M_{H}, and j is the index for the reagents X(j) that goes from 1 to N. We likewise obtain M_{D}N analogous results A_{D(i)X(j)} from the disease group, where i goes from 1 to M_{D}.

For each value of j we average the values of A_{H(i)X(j)} for i=1 to M_{H}:

$${A}_{HavX ( j )} = \frac{1}{{M}_{H}} {\sum }_{i = 1}^{{M}_{H}} {A}_{H ( i ) X ( j )} j = 1 , N \qquad ( 11 )$$

We likewise average the values of A_{D(i)X(j)} for each value of j:

$${A}_{DavX ( j )} = \frac{1}{{M}_{D}} {\sum }_{i = 1}^{{M}_{D}} {A}_{D ( i ) X ( j )} j = 1 , N \qquad ( 12 )$$

Similarly, for a corresponding set of Y(j) reagents (j=1 to N) we determine, by measurement or computation, or by a combination of measurement and computation as described above, values A_{H(i)Y(j)} for i=1 to M_{H}, and values A_{D(i)Y(j)} for i=1 to M_{D}. We compute average values for each value of j, for the M_{H} samples from healthy individuals and for the M_{D} samples from individuals with the disease:

$$\begin{matrix}{A}_{HavY ( j )} = \frac{1}{{M}_{H}} {\sum }_{i = 1}^{{M}_{H}} {A}_{H ( i ) Y ( j )} j = 1 , N & ( 13 ) \\ {A}_{DavY ( j )} = \frac{1}{{M}_{D}} {\sum }_{i = 1}^{{M}_{D}} {A}_{D ( i ) Y ( j )} j = 1 , N & ( 14 )\end{matrix}$$

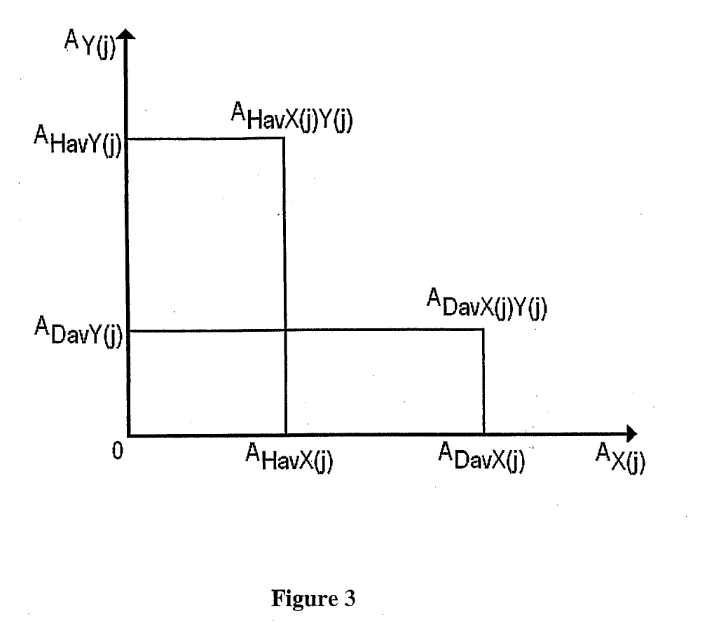

For a single pair of reagents X(j) and Y(j) and a given disease D we can plot the values A_{DavX(j)}, A_{HavX(j)}, A_{DavY(j)} and A_{HavY(j)} on the axes A_{X(j)} and A_{Y(j)} as shown in FIG. 2. Hence the points labelled A_{DavX(j)Y(j)} and A_{HavX(j)Y(j)} are defined for the average disease and average healthy states respectively. We need a stimulus that, firstly for this pair of reagents, moves the system from A_{DavX(j)Y(j)} towards A_{HavX(j)Y(j)}. An appropriate stimulus in the context of just X(j) and Y(j) then consists of two components, one for motion from right to left and one for motion upwards in the example shown in FIG. 3. The reagent Y(j) stimulates the complementary X(j) cells, and hence moves the system along the X(j) axis (the horizontal axis). The reagent X(j) stimulates Y(j) cells, and moves the system in the vertical axis. We next need to determine the appropriate concentrations of the reagents.

At first sight, we might choose a concentration of Y(j) proportional to A_{HavX(j)}−Ar_{DavX(j)} and a concentration of X(j) proportional to A_{HavY(j)}−Ar_{DavY(j)}. A problem with this is however that some of these tentative relative concentrations will be negative, and we cannot include a negative amount of a reagent in the formulation of a more complex reagent. This problem can be resolved by substituting a positive amount of the reagent X(j) for a negative amount of any reagent Y(j) [since X(j) is complementary to Y(j)], and likewise a positive amount of Y(j) for any negative amount of X(j). The relative amount of X(j) needed in the vaccine, from the perspective of the X(j)/Y(j) pair of reagents, will be denoted by R[X(j)] and is given by

$$R [ X ( j ) ] = [ {A}_{HavY ( j )} - {A}_{DavY ( j )} ] [ \frac{1 + sign ( {A}_{HavY ( j )} - {A}_{DavY ( j )} )}{2} ] + [ {A}_{HavX ( j )} - {A}_{DavX ( j )} ] [ \frac{1 - sign ( {A}_{HavX ( j )} - {A}_{DavX ( j )} )}{2} ] \qquad ( 15 )$$

where sign x=1 for x>0, and sign x=−1 for x<0. Similarly, the relative amount of Y(j) in the vaccine, denoted by R[Y(j)], is given by

$$R [ Y ( j ) ] = [ {A}_{HavX ( j )} - {A}_{DavX ( j )} ] [ \frac{1 + sign ( {A}_{HavX ( j )} - {A}_{DavX ( j )} )}{2} ] + [ {A}_{HavY ( j )} - {A}_{DavY ( j )} ] [ \frac{1 - sign ( {A}_{HavY ( j )} - {A}_{DavY ( j )} )}{2} ] \qquad ( 16 )$$

In the example of FIG. 3, both components in the expression for R[X(j)] are positive, and both components in the expression for R[Y(j)] are zero. The total composition of the vaccine is then obtained by summing over j. This is thus a method for formulating an immunogenic (vaccine) stimulus using the base set of N reagents. We then still have a single undetermined parameter, namely the ratio of the actual total concentration needed in the vaccine to the numerical values as computed. This parameter can be determined empirically by titration by one skilled in the art.

Immunizations with the X(j) and Y(j) reagents can also be delivered together with an adjuvant, which is an agent that non-specifically boosts immune responses to specific antigens.

### Application To Personally Customised Vaccines

People's individual antibody repertoires and/or T cell V region repertoires and/or B cell V region repertoires can be characterised as points in N-dimensional shape space using the present invention also while they are still healthy. Changes in their repertoire as they age can be monitored by measuring the similarity between current and historical samples from the same individual. Any undesired changes can then be counteracted at an early stage by the vaccine method of the invention. The preceding description is in terms of vaccines suitable for a particular disease and for many people. Such vaccines are applicable especially as a preventive immunisation for healthy people. A patient may however have skewing that is unique to that individual. In such cases a personally tailored approach is beneficial. One method is to replace the average absorbance values A_{DavX(j)} and A_{DavY(j)} with the patient's absorbance values A_{D(i)X(j)} and A_{D(i)Y(j)} respectively in equations (15) and (16). Another step in the direction of personally tailored vaccines is to replace A_{HavX(j)} with A_{H(i)X(j)} and A_{HavY(j)} with A_{H(i)Y(j)}, in equations (15) and (16), where A_{H(i)X(j)} and A_{H(i)Y(j)} are obtained using historical samples from when the individual i was healthy. Hence N-dimensional perturbations can be tailored to inhibit and/or reverse pathological skewing of V region repertoires at the levels of both populations and individuals.

### Other Applications

The Proteomic Analyser can be used to compare the repertoires of antibodies of young, healthy individual mice of different strains, and of different species. Hence it can be used to experimentally confirm that the repertoires of healthy young individuals of different strains and different species are similar to each other.

While the concept of using X(j)/Y(j) axis coordinates emerged in the context of the V region network of interactions of the immune system, this technology can be used generally to characterise proteomes and monitor changes in the proteome of an individual or an organism. A Proteomic Analyser point that does not include some of the components of a sample can be useful. For example, mapping the Proteomic Analyser point for immune system V regions, for example, for IgG antibodies, may require some purification of the antibodies. On the other hand, a Proteomic Analyser point that may usefully be monitored, and may have diagnostic value, could be one that includes all the serum components, or all the serum components except antibodies. Thus mapping of molecules other than immune system V regions in the N-dimensional shape space may also be useful in diagnostic applications.

The Proteomic Analyser can be used to measure similarity and dissimilarity in shapes between different proteins, including those for which a three dimensional structure is known and others for which a three dimensional structure is not known. It can thus be a tool that assists in the elucidation of the three dimensional structure of proteins. This in turn can assist in the design of drugs that interact with particular proteins.

The Proteomic Analyser can measure Proteomic Analyser points for both biological and non-biological samples. It can provide a method for quality control for simple substances or mixtures of substances that may be simple or complex.

### Preferred Embodiments

The invention utilises a diverse array of N reagents (N>>1) and the set of relative binding affinities of the substances for each other, as determined for example by an ELISA assay. A value of N in the range 20 to 1000 is anticipated, but the invention is not limited to this range. There is not a specific minimum value of N. From the perspective that the specificity of the method depends exponentially on the value of N (see below), the larger the value of N the better. From a practical point of view, the technology is likely to be at least initially implemented using ELISA plates that have 96, 384 or 1536 wells. A plausible implementation involves each plate containing N X(j) reagents and N Y(j) reagents, so that N is in the range of between about 40 and about 750. The choice of this range includes the possibility of using some of the wells as calibration controls. The use of other technologies for measuring the binding of reagents to each other and the binding of samples to the reagents may lead to other preferred values of N, that are specific to the details of those technologies.

The N reagents (X(j), j=1, N) are substances with reproducible, stable, diverse, three dimensional shapes and may include for example monoclonal antibodies and/or other proteins from one or more species. The invention optionally utilises also a second array of N reagents (Y(j), j=1, N), consisting of mixtures of the first array of N reagents, formulated as described in the above specification.

One preferred embodiment is for all the X(j) reagents to be monoclonal antibodies, for example all of the IgG class. This creates a symmetry in the system that allows for essentially unlimited diversity in shapes, while ensuring that all the reagents have a similar intrinsic ability to cross-link complementary receptors. (The cross-linking of receptors is believed to be the mechanism for the specific stimulation of lymphocytes.) This would be in contrast to using proteins with varying degrees of polymerisation, some of which would be much stronger immunogenic stimuli than others. IgG antibodies have two V regions, and are thus able to cross-link complementary receptors. Another preferred embodiment is to use exclusively soluble proteins of a size comparable to each other and without any repeating determinants, again ensuring that they are of similar immunogenicity.

The set of reagents should optimally have an essentially random interaction matrix K (or K^{P}). The randomness of K or K^{P} will correlate with the matrix J (or J^{P}) being diagonally dominant. This diagonal dominance of J in turn correlates with the shape space axes being approximately orthogonal to each other. Thus the degree of diagonal dominance of J can be used as a measure of quality for a candidate set of the reagents X(j) and, by extension the corresponding Y(j). In order to increase the fraction of nonzero terms in the interaction matrix K, the reagents X(j) can themselves be mixtures of reagents, for example mixtures of proteins or (more specifically) of monoclonal antibodies. If the diagonal terms in the matrix J all have approximately the same size, there is a high level of symmetry in the shape space, which is beneficial.

For applications of the Proteomic Analyser involving the binding of the reagents to V regions in serum samples, it may be necessary to purify the V region bearing molecules in order to decrease the noise due to binding of the reagents to non-V region bearing molecules. A preferred embodiment is to constrain the set of X(j) reagents such that they have minimal affinity for proteins in the samples being mapped except for the V regions in those samples.

### EXAMPLE

### SARS

We are currently faced with an important new disease, namely SARS. A virus has been identified as the culprit. But the virus is not found to be present in all cases of the disease. Several years ago this seemed to be the case with AIDS and HIV, but then cases of the syndrome that were negative for HIV were defined as “idiopathic CD4+ T-lymphocytopenia”, rather than AIDS. Smith et al. 1993, N. Engl. J. Med., 328, 373-379; Ho et al. 1993, N. Engl. J. Med., 328, 380-385; Spira et al. 1993, N. Engl. J. Med. 328, 386-392; Duncan et al. 1993, N. Engl. J. Med. 328, 393-398. The definition of AIDS was narrowed to include only those people who are positive for HIV. Morbidity and Mortality Weekly Report, CDC Atlanta, USA 1999, 48 (RR13), 1-31.

We may now have a similar situation with SARS. The World Health Organisation has announced that a corona virus has been shown to cause the disease (see http://www.who.int/mediacentre/releases/2003/pr31/en/) but in Canada only about 50% of confirmed SARS patients were found to be positive for direct detection of the virus, namely polymerase chain reaction or virus culture (Frank Plummer, personal communication). Ultimately, about 95% of confirmed cases developed antibody to SARS coronavirus at 4 weeks (Frank Plummer, personal communication). This raises the question of whether SARS can be caused by a proteomic stimulus similar to that caused by the virus, but without the virus itself The method described here may be useful for identifying any additional causes of SARS. Responses to the corona virus would produce one form of repertoire skewing, while other agents may induce a similar but distinct skewing. The invention potentially enables a diagnosis for SARS that is independent of the detection of the corona virus or any other virus.

### The Specificity of the Method And the Value of N

The specificity of the method depends on the value of N and the accuracy of the assay method. If the values of A_{iX(j)}−Ar_{iY(j)} are obtained simply as Boolean numbers, when N=20 the shape space would have 220 distinguishable points. With an ELISA assay the results are however analogue rather than Boolean, and each coordinate might have 10 distinguishable values. Then already with N=5 the shape space would have 10⁵ distinguishable points, and with N=20 there would be 10²⁰ distinguishable points. This theoretical remarkable resolution is expected to be important for applications to diagnostics and vaccines. It can be tested in experiments in which known mixtures of the X(j) reagents themselves are analysed using the method, and experimentally determined coordinates are compared with theoretical predictions.

### Relationship To Some Other Work On Shape Space

In their work on shape space Perelson et al. 1979 J. theoret. Biol. 81, 645-667, estimated limits on the size of the repertoire that is needed to reliably respond to antigen, and they were also concerned with the necessity not to make antibodies to self. The focus of the theory is the relationship between the volume of shape space covered by the reactivity of a single antibody and the total volume of shape space, and hence the number of different antibodies needed to reliably cover shape space. The main parameters in the theory are the dimension of their shape space N, the size of the repertoire N_{Ab}, and the distance in shape space within which an antibody can bind all antigens, ε. These parameters are interdependent, and the theory did not include a method for measuring N or ε. On the basis of literature values of the frequencies of antigen specific cells, they estimated that N could not be more than 5 or 10.

Lapedes et al., 2001, J. theor. Biol. 212, 57-69, described a shape space for which a dimensionality can be determined using experimental data. They used MN experimental data points, namely the binding of M antigens to N antisera, to map the shapes of each of the antigens and sera to points in a D-dimensional shape space. The method involves minimizing a function of the experimental data points and the space shape coordinates. The relationship of this shape space to that of Perelson et al. is unclear, since it does not have ε or N_{Ab} as parameters. They found D to have a value of 4 to 5.

These papers by Perelson et al. and Lapedes et al. are based on the premise that there is an intrinsic dimensionality for shape space relevant to immunological recognition. This premise plays no role in this invention.

This invention is an extension of and improvement on the earlier concept of serological distance coefficients, in which similarity was defined in the context of a single diverse reagent, Hoffmann et al., 1989. Immunol. Letters, 22, 83-90. Here we define similarity in the context of an approximately orthogonal set of N axes in shape space. In immunology context is of over-riding importance, since antibodies are made in the context of a set of self antigens, T cells and other antibodies. The dimension N of the shape space is something we are free to choose, and the choice determines the level of specificity. The larger the value of N, the higher the specificity of the method.

1. A method for mapping a sample i, in an N-dimensional shape space with orthogonal axes, where N is an integer, comprising:
(a) selecting a set of N reagents X(j) where j=1 to N;
(b) measuring a first binding signal for each of the N X(j) reagents binding to each other, to produce a matrix with elements Kⱼₖ (measured) and deriving from this matrix a symmetrical matrix K in which each element of K, namely Kⱼₖ, is equal to the larger of Kⱼₖ (measured) and Kₖⱼ (measured), (j=1 to N and k=1 to N);
(c) defining a set of N new reagents Y(j), where j=1 to N, as linear combinations of said X(j), with relative concentration of k^{th} components X(k) in Y(j) being proportional to Kⱼₖ for k=1 to N;
(d) establishing a symmetry between the X(j) reagents and the Y(j) reagents by one of:
i) making a total concentration of components of each of said Y(j) reagents such that a second binding signal obtained for Y(j) binding to X(j) is equal to a converse binding signal for X(j) binding to Y(j); and
ii) setting a total concentration of components of each of said Y(j) reagents equal to a constant C₀, wherein C₀ is a concentration of each of the X(j) reagents;
(e) measuring binding signals Aᵢₓ₍ⱼ₎ for each one of said X(j) reagents to substances in the sample i;
(f) measuring binding signals A_{iY(j)} (measured) for each one of said Y(j) reagents to substances in the sample i;
(g) normalizing said binding signals A_{iY(j)} (measured) such that an average of the binding signals A_{iY(j)} (measured) (j=1 to N) is the same as an average of the binding signals A_{iX(j)} (j=1 to N).
(h) computing N coordinates for the sample i as Aᵢⱼ=A_{iX(j)}−A_{iY(j)} (measured), j=1 to N.

2. A method for mapping a sample i, in an N-dimensional shape space with orthogonal axes, where N is an integer, comprising:
(a) selecting a set of N reagents X(j) where j=1 to N;
(b) measuring a first binding signal for each of the N reagents binding to each other, to produce a matrix K with elements Kⱼₖ (j=1 to N and k=1 to N);
(c) defining a set of N new reagents Y(j), where j=1 to N, as linear combinations of said X(j), with relative concentrations of k^{th} components X(k) in Y(j) being proportional to Kⱼₖ for k=1 to N;
(d) establishing a symmetry between the X(j) reagents and the Y(j) reagents by one of:
i) making a total concentration of components of each of said Y(j) reagents such that a second binding signal obtained for Y(j) binding to X(j) is equal to a converse binding signal for X(j) binding to Y(j); and
ii) setting a total concentration of components of each of said Y(j) reagents equal to a constant C₀, wherein C₀ is a concentration of each of the X(j) reagents;
(e) measuring binding signals A_{iX(j)} for each one of said X(j) reagents to substances in the sample i;
(f) computing binding signals A_{iY(j)} (expected) according to:
$${A}_{iY ( j )} ( expected ) ∝ {\sum }_{k = 1}^{N} {A}_{iX ( k )} {K}_{kj} ;$$
(g) normalizing said binding signals A_{iY(j)} (expected) so that an average of said binding signals A_{iY(j)} (expected) (j=1 to N) is the same as an average of said binding signals A_{iX(j)} (j=1 to N);
(h) computing N coordinates for the sample i according to: Aᵢⱼ=A_{iX(j)}−A_{iY(j)} (expected), j=1 to N.

3. A method for mapping a sample i in an N-dimensional shape space with orthogonal axes, where N is an integer, comprising:
(a) selecting a set of N reagents X(j) where j=1 to N;
(b) measuring a first binding signal for each of the N reagents binding to each other, to produce a matrix K with elements Kⱼₖ (j=1 to N and k=1 to N);
(c) defining a set of N new reagents Y(j), where j=1 to N, as linear combinations of said X(j), with relative concentrations of k^{th} components X(k) in Y(j) being proportional to Kⱼₖ for k=1 to N;
(d) establishing a symmetry between the X(j) reagents and the Y(j) reagents by one of:
i) making a total concentration of components of each of said Y(j) reagents such that a second binding signal obtained for Y(j) binding to X(j) is equal to a converse binding signal for X(j) binding to Y(j); and
ii) setting a total concentration of components of each of said Y(j) reagents equal to a constant C₀,wherein C₀ is a concentration of each of the X(j) reagents;
(e) measuring binding signals A_{iX(j)} for each one of said X(j) reagents to substances in the sample i;
(f) measuring binding signals A_{iY(j)} (measured) for each one of said Y(j) reagents to substances in the sample i;
(g) normalizing said binding signals A_{iY(j)} (measured) so that an average of the binding signals A_{iY(j)} (measured) (j=1 to N) is the same as an average of the binding signals A_{iX(j)} (j=1 to N);
(h) computing binding signals A_{iY(j)} (expected) according to:
$${A}_{iY ( j )} ( expected ) ∝ {\sum }_{k = 1}^{N} {A}_{iX ( k )} {K}_{kj} ;$$
(i) normalizing said binding signals A_{iY(j)} (expected) such that an average of the binding signals A_{iY(j)} (expected) (j=1 to N) is the same as an average of the binding signals A_{iX(j)} (j=1 to N);
(j) computing binding signals A_{iY(j)} (mean) according to: A_{iY(j)} (mean)=0.5*[A_{iY(j)} (measured)+A_{iY(j)} (expected)];
(k) computing N coordinates for the sample i as Aᵢⱼ=A_{iX(j)}−A_{iY(j)} (mean), j=1 to N.

4. A method for mapping a sample i, in an N-dimensional shape space with orthogonal axes, where N is an integer, comprising:
(a) selecting a set of P reagents X(j) where P>N and j=1 to P;
(b) measuring a first binding signal for each of the reagents X(j) binding to each other, to produce a P×P matrix K^{P} (measured) with elements “K^{P}ⱼₖ (measured)” (with j=1 to P and k=1 to P), and deriving from said matrix K^{P} (measured) a symmetrical matrix K^{P} in which each element, namely K^{P}ⱼₖ, is equal to the larger of K^{P}ⱼₖ (measured) and K^{P}ₖⱼ (measured), (j=1 to P and k=1 to P);
(c) formulating a set of P reagents Y(j), where j=1 to P, as linear combinations of said reagents X(j), with relative concentrations of k^{th} components X(k) in Y(j) being proportional to K^{P}ⱼₖ, for k=1 to P;
(d) measuring a second binding signal for each of the X(j) reagents binding to each of the Y(j) reagents, to produce a P×P matrix “J^{P}” with elements “J^{P}ⱼₖ” (j=1 to P and k=1 to P);
(e) selecting N X(j) and N Y(j) reagents having largest ratios of diagonal elements of J^{P} to a mean of corresponding off-diagonal elements;
(f) establishing a symmetry between the X(j) reagents and the Y(j) reagents by one of:
i) making a total concentration of components of each of said Y(j) reagents such that a second binding signal obtained for Y(j) binding to X(j) is equal to a converse binding signal for X(j) binding to Y(j); and
ii) setting a total concentration of components of each of said Y(j) reagents equal to a constant C₀, wherein C₀ is a concentration of each of the X(j) reagents;
(g) measuring binding signals A_{iX(j)} for each one of said X(j) reagents to substances in the sample i;
(h) determining binding signals A_{iY(j)} as described in one of:
i) steps ((j) and (g) of claim 1 and said binding signals A_{iY(j)}=A_{iY(j)} (measured);
ii) steps (b) and (c) of claim 2 wherein Kⱼₖ is replaced by K^{P}ⱼₖ (for j=1 to N and k=1 to P) and said binding signals A_{iY(j)}=A_{iY(j)} (expected); and
iii) steps (b) to (f) of claim 3 wherein Kⱼₖ is replaced by K^{P}ⱼₖ (for j=1 to N and k=1 to P) and said binding signals A_{iY(j)} =A_{iY(j)} (mean);
(i) computing N coordinates for the sample i as Aᵢⱼ=A_{iX(j)}−A_{iY(j)}, j=1 to N.

5. A method for classifying a sample U with respect to Q categories, wherein Q is equal to or greater than 2, and wherein each of the categories Q is identified by a value of q where q=1 to Q, the method comprising:
(a) selecting M_{q} samples known by conventional criteria to belong to each one of said categories q;
(b) for each one of said categories q mapping said samples M_{q} in an N-dimensional shape space using the method of claim 1, claim 2, claim 3 or claim 4, giving coordinates A_{qij} with q=1 to Q, i=1 to M_{q} and j=1 to N, said coordinates A_{qij} denoted by A_{qi};
(c) mapping said sample U in the N-dimensional shape space using the method of claim 1, claim 2, claim 3, or claim 4 giving coordinates A_{Uj}, with j=1 to N, said coordinates A_{Uj} denoted by A_{U};
(d) for each one of said q categories computing N average coordinates A_{qav(j)} for j=1 to N and q=1 to Q, of the M_{q} samples according to:
$${A}_{qav ( j )} = \frac{1}{{M}_{q}} {\sum }_{i = 1}^{{M}_{q}} {A}_{qij}$$
said average coordinates A_{qav(j)} denoted by A_{qav}, with q=1 to Q;
(e) selecting two average coordinates A_{qav} to define a new axis in shape space, wherein a first average coordinate A_{qav} for a first one of said categories is denoted by A₁ₐᵥ and wherein a second average coordinate A_{qav} for a second one of said categories is denoted by A₂ₐᵥ wherein said first and second average coordinates A₁ₐᵥ and A₂ₐᵥ each have N coordinates A₁ₐᵥ₍ⱼ₎ and A₂ₐᵥ₍ⱼ₎, respectively, with j=1 to N;
(f) calculating a Euclidean distance between the first and second average coordinates A₁ₐᵥ and A₂ₐᵥ, wherein said distance is denoted by c, according to:
$$c = \sqrt{{\sum }_{j = 1}^{N} {( {A}_{1 av ( j )} - {A}_{2 av ( j )} )}^{2}} ;$$
(g) computing xᵢ for all Aᵢ according to:
$${x}_{i} = \frac{1}{2 c} ( {a}_{i}^{2} - {b}_{i}^{2} + {c}^{2} )$$
wherein A_{qi} and A_{U} are collectively referred to as Aᵢ, a Euclidean distance from each Aᵢ to A₁ₐᵥ is designated aᵢ, a Euclidean distance from each Aᵢ to A₂ₐᵥ is designated bᵢ, and wherein Eᵢ designates a point of intersection between a line and a A₁ₐᵥ/A₂ₐᵥ axis, said line extending from Aᵢ to said A₁ₐᵥ/A₂ₐᵥ axis at right angles to the A₁ₐᵥ/A₂ₐᵥ axis, and wherein xᵢ denotes a distance from A₁ₐᵥ to Eᵢ;
(h) computing a mean and standard deviation of the xᵢ for samples in the first category and the second category, said means denoted by μ₁(xᵢ) and μ₂(xᵢ) respectively, and said standard deviations denoted by σ₁(xᵢ) and σ₂(xᵢ), respectively;
(i) calculating a z statistic, z_{U(q)}, for the xᵢ of the unclassified sample U relative to the distribution of xᵢ values for samples in each of the first and second categories,
$${z}_{U ( q )} = \frac{{x}_{i} ( U ) - {μ}_{q} ( {x}_{i} )}{{σ}_{q} ( {x}_{i} )}$$
wherein xᵢ(U) denotes a value of xᵢ for the unclassified sample; and
(j) determining from the z statistic a level of confidence with which the unclassified sample U can be excluded from the first and second categories.

6. A method for classifying a sample U with respect to Q categories, wherein Q is equal to or greater than 2, and wherein each of the categories Q is identified by a value of q where q=1 to Q, the method comprising the following steps:
(a) steps (a) to (d) of claim 5;
(b) computing standard deviations σ_{qj}(j=1 to N, q=1, Q) for each of the N coordinates of the M_{q} samples according to:
$${σ}_{qj} = \sqrt{\frac{{\sum }_{i = 1}^{{M}_{q}} {( {A}_{qij} - {A}_{qav ( j )} )}^{2}}{{M}_{q} - 1}}$$
(c) computing estimates of a ratio [P_{U1}/P_{U2}]ⱼ of a probability that the unclassified sample U belongs to a first one of said categories, to a probability that the sample U belongs to a second one of said categories, based on the data for the j^{th} shape space axis, according to:
$$F ( {A}_{U ( j )} , {A}_{1 av ( j )} , {σ}_{1 j} ) = \frac{1}{{σ}_{1 j} \sqrt{2 π}} exp ( - \frac{1}{2} {( \frac{{A}_{Uj} - {A}_{1 av ( j )}}{{σ}_{1 j}} )}^{2} )$$
$$F ( {A}_{Uj} , {A}_{2 av ( j )} , {σ}_{2 j} ) = \frac{1}{{σ}_{2 j} \sqrt{2 π}} exp ( - \frac{1}{2} {( \frac{{A}_{U j} - {A}_{2 av ( j )}}{{σ}_{2 j}} )}^{2} ) , \\ {and [ {P}_{U 1} / {P}_{U 2} ]}_{j} = \frac{F ( {A}_{Uj} , {A}_{1 av ( j )} , {σ}_{1 j} )}{F ( {A}_{Uj} , {A}_{2 av ( j )} , {σ}_{2 j} )} ; for j = 1 to N , and$$
(d) computing a joint probability ratio, [P_{U1}/P_{U2}]_{all N axes}, as a product from j=1 to j=N of probabilities for each axis [P_{U1}/P_{U2}]ⱼ.

7. The method of claim 5, wherein Q≧3 and steps (e) to (j) are repeated so as to determine levels of confidence with which the sample U can be excluded from further categories.

8. The method of claim 6, wherein Q≧3 and steps (c) and (d) are repeated so as to determine ratios of probabilities for the sample U belonging to further categories.

9. The method of claim 5, 6, 7 or 8, wherein said samples are biological samples taken from vertebrates and said categories include samples from one or more healthy vertebrates, diseased vertebrates, and vertebrates predisposed to develop disease.

10. A method for predicting development of a disease in a vertebrate, comprising:
(a) producing a set of N-dimensional shape space coordinates by mapping in an N-dimensional shape space each one of a plurality of biological samples obtained from the vertebrate at multiple points in time, using the method of claim 1, claim 2 claim 3 or claim 4;
(b) determining positions of said coordinates relative to an N-dimensional vector from a first point in the N-dimensional shape space to a second point in the N-dimensional shape space, wherein said first point is characteristic of vertebrates without said disease and said second point is characteristic of vertebrates with said disease; and
(c) determining whether said coordinates are moving with time from said first point towards said second point in the N-dimensional shape space, wherein movement from said first point towards said second point indicates a progression toward said disease.

11. A method for preventing the development of a disease in a vertebrate comprising:
(a) selecting and establishing a symmetry for a set of N reagents X(j) and N reagents Y(j) using one of:
i) steps (a) to (d) of claim 1; and
ii) steps (a) to (f) of claim 4;
(b) measuring binding signals A_{H(i)X(j)} for each X(j) reagent to immune system V regions in samples H(i), for i=1 to M_{H} and j=1 to N, where samples H(i) are biological samples from each one of M_{H} healthy vertebrates, and determining binding signals A_{D(i)X(j)} for each X(j) reagent to immune system V regions in samples D(i), for i=1 to M_{D} and j=1 to N, where samples D(i) are biological samples from each one of M_{D} vertebrates classified as having the disease;
(c) determining binding signals A_{H(i)Y(j)} for each Y(j) reagent to immune system V regions in the samples H(i), (i=1 to M_{H} and j=1 to N), and binding signals A_{D(i)Y(j)} for each Y(j) reagent to immune system V regions in the samples D(i), (i=1 to M_{D} and j=1 to N), by one of the following:
i) measuring binding signals of each of the Y(j) reagents to the samples H(i), (i=1 to M_{H} and j=1 to N) and to the samples D(i), (i=1 to M_{D} and j=1 to N), and normalizing said binding signals A_{H(i)Y(j)} and A_{D(i)Y(j)} so that an average of said binding signals A_{H(i)Y(j)} is equal to an average of said binding signals A_{H(i)X(j)} and an average of said binding signals A_{D(i)Y(j)} (j=1, N) is equal to an average of said binding signals A_{D(i)X(j)} (j=1, N);
ii) steps (b) to (c) of claim 2;
iii) steps (b) to (f) of claim 3;
iv) steps (b) and (c) of claim 2, wherein Kⱼₖ is replaced by K^{P}ⱼₖ (for j=1 to N and k=1 to P); and
v) steps (b) to (f) of claim 3 wherein Kⱼₖ is replaced by K^{P}ⱼₖ (for j=1 to N and k=1 to P);
(d) computing average values of A_{H(i)X(j)}, A_{H(i)Y(j)}, A_{D(i)X(j)} and A_{D(i)Y(j)}, namely A_{HavX(j)}, A_{HavY(j)}, A_{DavX(j)} and A_{DavY(j)} respectively, according to:
$${A}_{HavX ( j )} = \frac{{\sum }_{i = 1}^{{M}_{H}} {A}_{H ( i ) X ( j )}}{{M}_{H}}$$
$${A}_{HavY ( j )} = \frac{{\sum }_{i = 1}^{{M}_{H}} {A}_{H ( i ) Y ( j )}}{{M}_{H}}$$
$${A}_{DavX ( j )} = \frac{{\sum }_{i = 1}^{{M}_{D}} {A}_{D ( i ) X ( j )}}{{M}_{D}}$$
$${A}_{DavY ( j )} = \frac{{\sum }_{i = 1}^{{M}_{D}} {A}_{D ( i ) Y ( j )}}{{M}_{D}} ;$$
(e) vaccinating the vertebrate with a vaccine containing the X(j) and Y(j) reagents, (j=1 to N), wherein relative amounts of the X(j) and Y(j) reagents in said vaccine are determined according to:
$$R [ X ( j ) ] = [ {A}_{HavY ( j )} - {A}_{DavY ( j )} ] [ \frac{1 + sign ( {A}_{HavY ( j )} - {A}_{DavY ( j )} )}{2} ] + [ {A}_{HavX ( j )} - {A}_{DavX ( j )} ] [ \frac{1 - sign ( {A}_{HavX ( j )} - {A}_{DavX ( j )} )}{2} ] \\ R [ Y ( j ) ] = [ {A}_{HavX ( j )} - {A}_{DavX ( j )} ] [ \frac{1 + sign ( {A}_{HavX ( j )} - {A}_{DavX ( j )} )}{2} ] + [ {A}_{HavY ( j )} - {A}_{DavY ( j )} ] [ \frac{1 - sign ( {A}_{HavY ( j )} - {A}_{DavY ( j )} )}{2} ] .$$

12. A method for treating a disease in a vertebrate, comprising:
(a) selecting a set of N reagents X(j) and N reagents Y(j) using one of:
i) steps (a) to (d) of claim 1; and
ii) steps (a) to (f) of claim 4;
(b) measuring binding signals A_{H(i)X(j)} for each X(j) reagent to immune system V regions in the samples H(i), for i=1 to M_{H} and j=1 to N, where samples H(i) are biological samples from each one of M_{H} healthy vertebrates, wherein i=1 to M_{H}, and determining binding signals A_{D(i)Y(j)} for each X(j) reagent to immune system V regions in the samples D(i), for i=1 to M_{D} and j=1 to N, where samples D(i) are biological samples from each one of M_{D} vertebrates classified as having the disease, wherein i=1 to M_{D};
(c) determining binding signals A_{H(i)Y(j)} for each Y(j) reagent to immune system V regions in the samples H(i), (i=1 to M_{H} and j=1 to N), and binding signals A_{D(i)Y(j)} for each Y(j) reagent to immune system V regions in the samples D(i), (i=1 to M_{D} and j=1 to N) by one of the following:
i) measuring binding signals of each of the Y(j) reagents to the samples H(i), (i =1 to M_{H} and j=1 to N) and to the samples D(i), (i=1 to M_{D} and j=1 to N), and normalizing said binding signals A_{H(i)Y(j)} and A_{D(i)Y(j)} so that an average of said binding signals A_{H(i)Y(j)} is equal to an average of said binding signals A_{H(i)X(j)} and an average of said binding signals A_{D(i)Y(j)} (j=1, N) is the same as an average of said binding signals A_{D(i)X(j)} (j=1, N);
ii) steps (b) to (c) of claim 2;
iii) steps (b) to (f) of claim 3;
iv) steps (b) and (c) of claim 2, wherein Kⱼₖ is replaced by K^{P}ⱼₖ (for j=1 to N and k=1 to P); and
v) steps (b) to (f) of claim 3 wherein Kⱼₖ is replaced by K^{P}ⱼₖ (for j=1 to N and k=1 to P);
(d) computing average values of A_{H(i)X(j)}, A_{H(i)Y(j)}, A_{D(i)X(j)} and A_{D(i)Y(j)}, namely, A_{HavX(j)}, A_{HavY(j)}, A_{DavX(j)}and A_{DavY(j)} respectively, according to:
$${A}_{HavX ( j )} = \frac{{\sum }_{i = 1}^{{M}_{H}} {A}_{H ( i ) X ( j )}}{{M}_{H}}$$
$${A}_{HavY ( j )} = \frac{{\sum }_{i = 1}^{{M}_{H}} {A}_{H ( i ) Y ( j )}}{{M}_{H}}$$
$${A}_{DavX ( j )} = \frac{{\sum }_{i = 1}^{{M}_{D}} {A}_{D ( i ) X ( j )}}{{M}_{D}}$$
$${A}_{DavY ( j )} = \frac{{\sum }_{i = 1}^{{M}_{D}} {A}_{D ( i ) Y ( j )}}{{M}_{D}} ;$$
(e) immunizing the vertebrate with a vaccine containing reagents X(j) and Y(j), (j=1 to N), relative amounts of X(j) and Y(j) in said vaccine being given by:
$$R [ X ( j ) ] = [ {A}_{HavY ( j )} - {A}_{DavY ( j )} ] [ \frac{1 + sign ( {A}_{HavY ( j )} - {A}_{DavY ( j )} )}{2} ] + [ {A}_{HavX ( j )} - {A}_{DavX ( j )} ] [ \frac{1 - sign ( {A}_{HavX ( j )} - {A}_{DavX ( j )} )}{2} ] \\ R [ Y ( j ) ] = [ {A}_{HavX ( j )} - {A}_{DavX ( j )} ] [ \frac{1 + sign ( {A}_{HavX ( j )} - {A}_{DavX ( j )} )}{2} ] + [ {A}_{HavY ( j )} - {A}_{DavY ( j )} ] [ \frac{1 - sign ( {A}_{HavY ( j )} - {A}_{DavY ( j )} )}{2} ] .$$

13. The method of claim 11 or 12 wherein said method is customized for a specific vertebrate by having A_{DavX(j)} and A_{DavY(j)} in the expressions for R[X(j)] and R[Y(j)] replaced by corresponding values for said specific vertebrate, namely, A_{D(i)X(j)} and A_{D(i)Y(j)}.

14. The method of claim 11, 12 or 13, wherein A_{HavX(j)} and A_{HavY(j)} are replaced by A_{H(i)X(j)} and A_{H(i)Y(j)}, where A_{H(i)X(j)} and A_{H(i)Y(j)} are obtained using historical samples from the vertebrate when the vertebrate was healthy.

15. The method of claim 10, 11, 12, 13 or 14 wherein the disease is an autoimmune disease or cancer.

16. The method of claim 10, 11, 12, 13, 14 or 15 wherein the vertebrate is a human.

17. The method of claim 1, 2, 3 or 4 wherein said X(j) reagents are substances that have diverse three-dimensional shapes.

18. The method of claim 1, 2, 3 or 4, wherein the X(j) reagents include proteins.

19. The method according to any one of claims 1 to 18, wherein said reagents X(j) are antibodies.

20. The method of claim 5, 6, 7 or 8, wherein said first category is a reference category.

21. A plate for use in classification or analysis of samples, said plate comprising 2N wells, wherein a first group of N wells are each coated with one of N reagents X(j) and a second group of N wells are each coated with one of N reagents Y(j), where N>>1, and the Y(j) reagents are mixtures of the X(j) reagents wherein relative concentrations of k^{th} components X(k) in each reagent Y(j) are proportional to Kⱼₖ, where Kⱼₖ is a binding signal for binding of X(j) to X(k), with j=1 to N and k=1 to N.

22. A set of reagents for use in classification or analysis of samples, medical diagnosis, therapeutic treatment of disease, or vaccination, said set of reagents comprising 2N reagents, wherein said set of reagents is made up of N X(j) reagents and N Y(j) reagents, wherein said Y(j) reagents are linear combinations of said X(j) reagents such that concentrations of k^{th} components of Y(j) are proportional to binding signals of X(j) to X(k), wherein j=1 to N and k=1 to N, wherein together said X(j) reagents and said Y(j) reagents define an orthogonal set of axes in shape space.

23. A plate for use in classification or analysis of samples, said plate comprising 2N wells, wherein a first group of N wells are each coated with one of N reagents X(j) and a second group of N wells are each coated with one of N reagents Y(j), where N>>1, and the Y(j) reagents are mixtures of the X(j) reagents wherein relative concentrations of k^{th} components X(k) in each reagent Y(j) are proportional to Kⱼₖ, where Kⱼₖ is a binding signal for binding of X(j) to X(k), with j=1 to N and k=1 to P, where P>N.

24. A set of reagents for use in classification or analysis of samples, medical diagnosis, therapeutic treatment of disease, or vaccination, said set of reagents comprising 2N reagents, wherein said set of reagents is made up of N X(j) reagents and N Y(j) reagents, wherein said Y(j) reagents are linear combinations of said X(j) reagents such that concentrations of k^{th} components of Y(j) are proportional to binding signals of X(j) to X(k) j=1 to N and k=1 to P, where P>N, wherein together said X(j) reagents and said Y(j) reagents define an orthogonal set of axes in shape space.

25. A set of reagents according to claim 22 or 24, wherein said binding signals are measured by an ELISA or RIA assay.
